(19) United States
(12) Patent Application Publication
(10) Pub. No.: US 2021/0166575 A1
(43) Pub. Date: Jun. 3, 2021

(54) SIMULATED TEXTURE PRESENTATION DEVICE, SIMULATED TEXTURE PRESENTATION METHOD, AND PROGRAM

(71) Applicant: NIPPON TELEGRAPH AND TELEPHONE CORPORATION, Tokyo (JP)

(72) Inventors: Mana SASAGAWA, Musashino-shi, Tokyo (JP); Arinobu NIIJIMA, Musashino-shi, Tokyo (JP); Ryosuke AOKI, Musashino-shi, Tokyo (JP); Tomoki WATANABE, Musashino-shi, Tokyo (JP); Tomohiro YAMADA, Musashino-shi, Tokyo (JP)

(73) Assignee: NIPPON TELEGRAPH AND TELEPHONE CORPORATION, Tokyo (JP)

(21) Appl. No.: 17/267,898

(22) PCT Filed: Jul. 9, 2019
(86) PCT No.: PCT/JP2019/027144

§ 371 Date: Feb. 11, 2021

(30) Foreign Application Priority Data
Aug. 13, 2018 (JP) 2018-152346

(51) Int. Cl. G09B 9/00

(52) U.S. Cl. CPC G09B 9/00

(57) ABSTRACT

A pseudo-food texture presentation device includes: an enclosure body in which a granular material is enclosed; a measurer configured to measure at least one index among a temperature of an ambient environment of the enclosure body, a humidity of the ambient environment of the enclosure body, a temperature of the enclosure body, and a moisture amount on a surface of the enclosure body; and an enclosure body controller configured to control density of the granular material inside the enclosure body in accordance with the measured index.

### TECHNICAL FIELD

The present invention relates to a pseudo-food texture presentation device, a pseudo-food texture presentation method, and a program.

### BACKGROUND ART

Studies on how food texture is presented have been conducted and applications to mastication training, entertainment, food design, and the like have been considered (for example, see NPL 1. Food texture is a physical property of food felt in the mouth. Accordingly, in order to present food texture, it is important to present the hardness and shape of food in the mouth of a user.

As a technique of the related art, a scheme of using a physical phenomenon such as a jamming transition to present hardness and a shape can be exemplified. The jamming transition is a physical phenomenon in which a granular material behaves differently depending on the density, and is a phenomenon in which a granular material behaves like a solid when the density is high and behaves like a fluid when the density is low.

As an example of a study in which a jamming transition is utilized, a technique applied to a robotic hand has been proposed. For example, NPL 2 proposes a scheme) of deforming a robotic hand in. accordance with the shape of an object by gripping the object which is in a robotic hand which is formed of a granular material and in a soft state so that the robotic hand is familiar with the shape of the object, and thus the object is gripped in a state in which the robotic hand is in a hard state.

### CITATION LIST

### Non Patent Literature

[NPL 1] Hiroo Iwata, Hiroaki Yano, Takahiro Uemura, and Tetsuro Moriya. 2004. Food Simulator: A Haptic Interface for Biting. In Proc. of VP. '04. 51-57.

[NPL 2] Eric Brown, Nicholas Rodenberg, John Amend, Annan Mozeika, Erik Steitz, Mitchell R. Zakin, Hod Lipson, and Heinrich M. Jaeger. 2010. Universal robotic gripper based on the jamming of granular material. Proc. of the National Academy of Sciences 107, 44 (2010), 18809-18814.

### SUMMARY OF THE INVENTION

### Technical Problem

In the content disclosed in NPL 1, it is difficult to present hardness and a shape in a mouth.

On the other hand, NPL 2 discloses a scheme of gripping an object depending on only whether a jamming transition arises or not. However, a technique for controlling hardness of an object in a user's mouth using the jamming transition has not been disclosed.

The present invention has been devised in view of the foregoing problems and an objective of the present invention is to provide a pseudo-food texture presentation device, a pseudo-food texture presentation method, and a program capable of controlling hardness of an object in a user's mouth using a jamming transition to present food texture by presenting hardness and a shape of the object.

### Means for Solving the Problem

According to a first aspect. of the present invention, a pseudo-food texture presentation device includes: an enclosure body in which a granular material is enclosed; a measurer configured to measure at least one index among a temperature of an ambient environment of the enclosure body, a humidity of the ambient environment of the enclosure body, a temperature of the enclosure body, and a moisture amount on a surface of the enclosure body; and an enclosure body controller configured to control density of the granular material inside the enclosure body in accordance with the measured index.

According to a second aspect of the present invention, in the pseudo-food texture presentation device in the first aspect, the enclosure body controller controls the density of the granular material inside the enclosure body when the measured index exceeds a preset threshold.

According to a third aspect of the present invention, in the pseudo-food texture presentation device in the first or second aspect, the enclosure body controller controls the density of the granular material inside the enclosure body in accordance with a cumulative value obtained by accumulating the measured index.

According to a fourth aspect of the present invention, the pseudo-food texture presentation device in any one of the first to third aspects further includes a determiner configured to determine whether the enclosure body is in a user's mouth. The enclosure body controller controls the density of the granular material inside the enclosure body in accordance with the measured index when the determiner determines that the enclosure body is in a user's mouth.

### Effects of the Invention

In the first aspect of the present invention, the density of the granular material inside the enclosure body is controlled in accordance with at least one index among the temperature of the ambient environment of the enclosure body, the humidity of the ambient environment of the enclosure body, the temperature of the enclosure body, and the moisture amount on the surface of the enclosure body. The hardness of the enclosure body is changed by a jamming transition by changing the density of the granular material inside the enclosure body. For example, when the enclosure body is in a user's mouth, the index is changed over time. In the foregoing configuration, it is possible to change the hardness of the enclosure body in the mouth of the user. As a result, it is possible to present the user with a pseudo-food texture.

In the second aspect of the present invention, the hardness of the enclosure body is changed before and after the measured value of the index exceeds the threshold. Thus, it is possible to change the hardness of the enclosure body step by step. When many thresholds are set, the hardness of the enclosure body can be smoothly changed and a diversity of the food textures can be improved.

In the third aspect of the present invention, the hardness of the enclosure body is changed in accordance with the cumulative value of the measured index. Thus, it is possible to gradually reduce the hardness of the enclosure body or increase the hardness of the enclosure body reliably.

In the fourth aspect of the present invention, the hardness of the enclosure body is changed in accordance with the measured value of the index and when the enclosure body is in the user's mouth. Thus, the hardness of the enclosure body can be changed by considering not only the measured value of the index but also a time that has passed after the user has put the enclosure body in her or his mouth. As a result, a diversity of food textures can be improved. A time at which the user is determined to have put the enclosure body in her or his mouth can be used as a standard time at which the measured values of the index are accumulated.

That is, according to the aspects of the present invention, it is possible to provide a technique capable of controlling hardness of an object in a user's mouth using a lamming transition. to present food texture by presenting hardness and a shape of the object.

### BRIEF DESCRIPTION OF DRAWINS

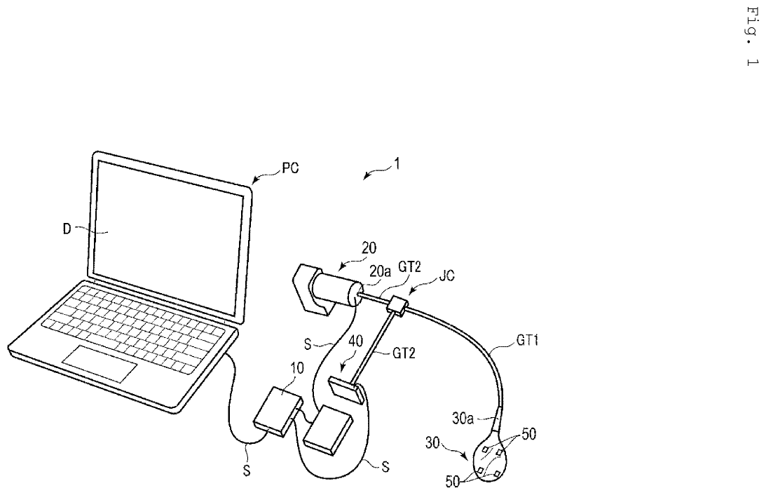

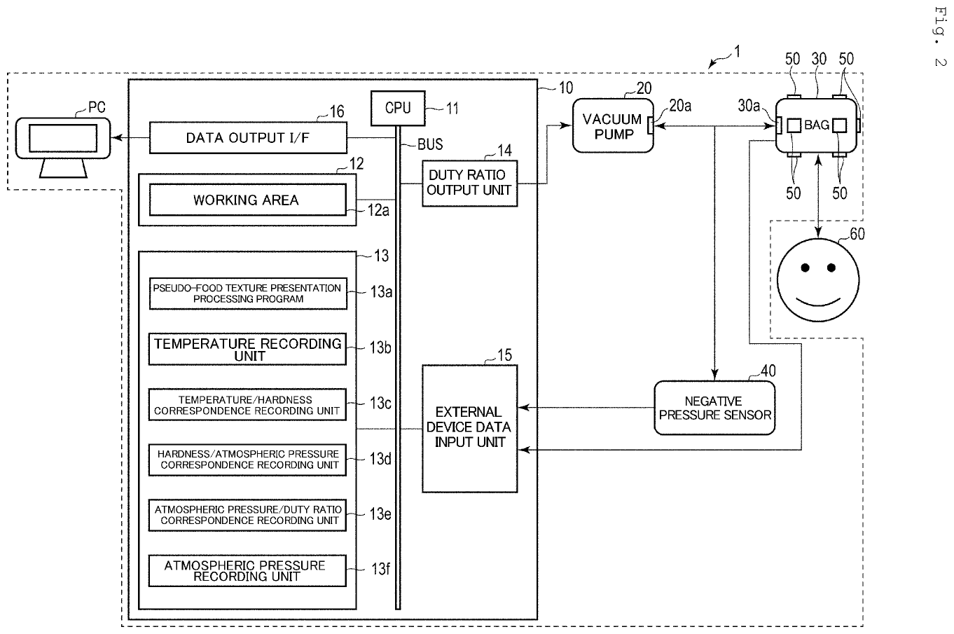

FIG. 1 is a perspective view illustrating an outer appearance of a pseudo-food texture presentation device according to First embodiment.

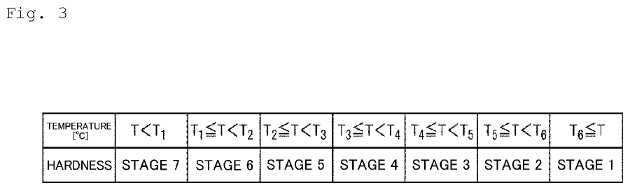

FIG. 2 is a block diagram illustrating a configuration of a microcomputer in the pseudo-food texture presentation device illustrated in FIG. 1.

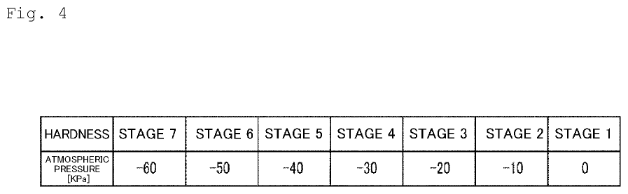

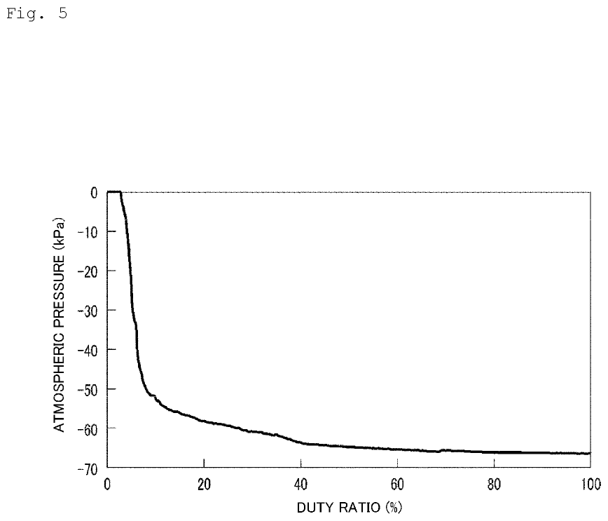

FIG. 3 is a diagram illustrating an example of a database recorded on a temperature/hardness correspondence recording unit illustrated in FIG. 2.

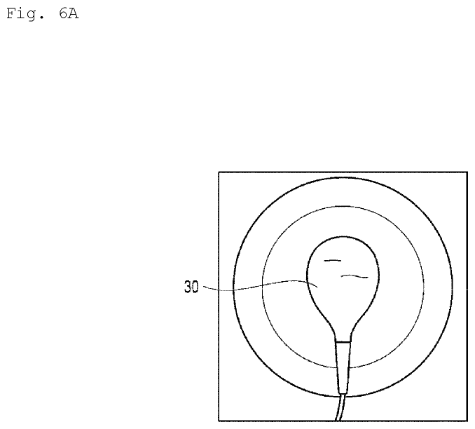

FIG. 4 is a diagram illustrating an example of a database recorded on a hardness/atmospheric pressure correspondence recording unit illustrated in FIG. 2.

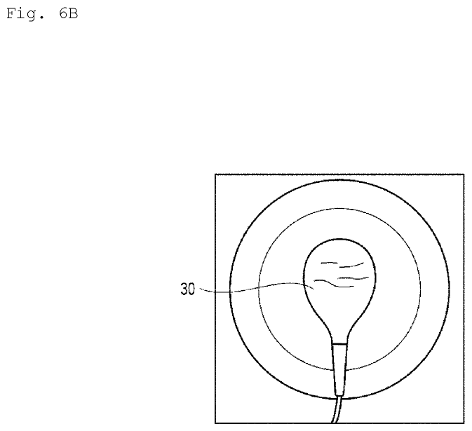

FIG. 5 is a diagram illustrating an example of a database recorded on an atmospheric pressure/duty ratio correspondence recording unit illustrated in FIG. 2.

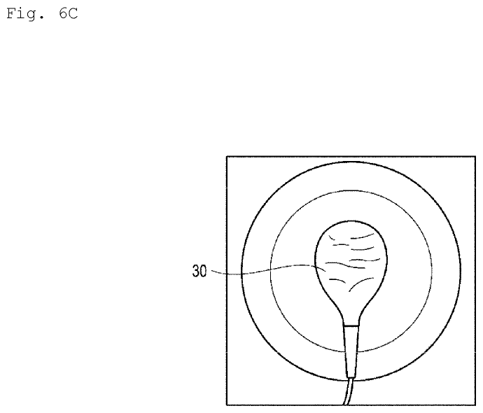

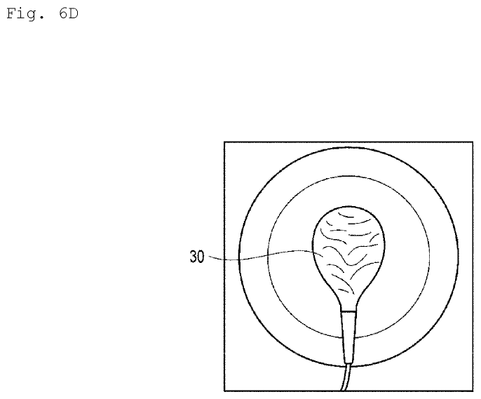

FIG. 6A is a diagram illustrating an aspect in which an atmospheric pressure inside a bag illustrated in FIG. 1 is adjusted to 0 [kPa].

FIG. 6B is a diagram illustrating an aspect in which an atmospheric pressure inside the bag illustrated in FIG. 1 is adjusted to −10 [kPa].

FIG. 6C is a diagram illustrating an aspect in which an atmospheric pressure inside the bag illustrated in FIG. 1 is adjusted to −30 [kPa].

FIG. 6D is a diagram illustrating an aspect in which an atmospheric pressure inside the bag illustrated in FIG. 1 is adjusted to −60 [kPa].

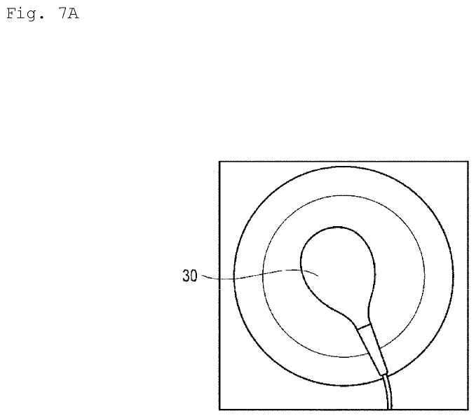

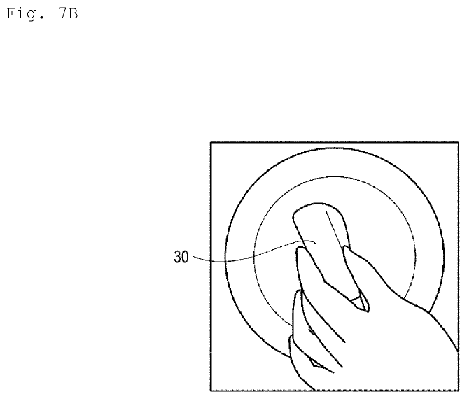

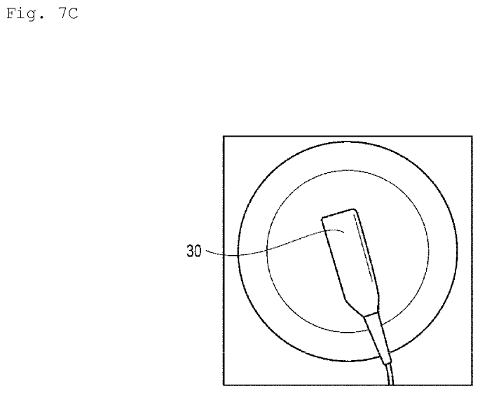

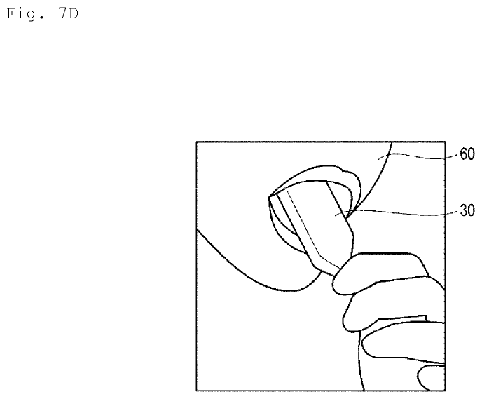

FIG. 7A is a diagram. illustrating a method of changing the shape of the bag illustrated in FIG. 1 and presenting the changed shape of the bag to a user.

FIG. 7B is a diagram illustrating a method of changing the shape of the bag illustrated in FIG. 1 and presenting the changed shape of the bag to the user.

FIG. 7C is a diagram illustrating a method of changing the shape of the bag illustrated in FIG. 1 and presenting the changed shape of the bag to the user.

FIG. 7D is a diagram illustrating a method of changing the shape of the bag illustrated in FIG. 1 and presenting the changed shape of the bag to the user.

FIG. 8 is a block diagram illustrating pseudo-food texture presentation processing realized by the pseudo-food texture presentation device illustrated in FIG. 2.

FIG. 9 is a block diagram illustrating a configuration of a pseudo-food texture presentation device according to Second embodiment.

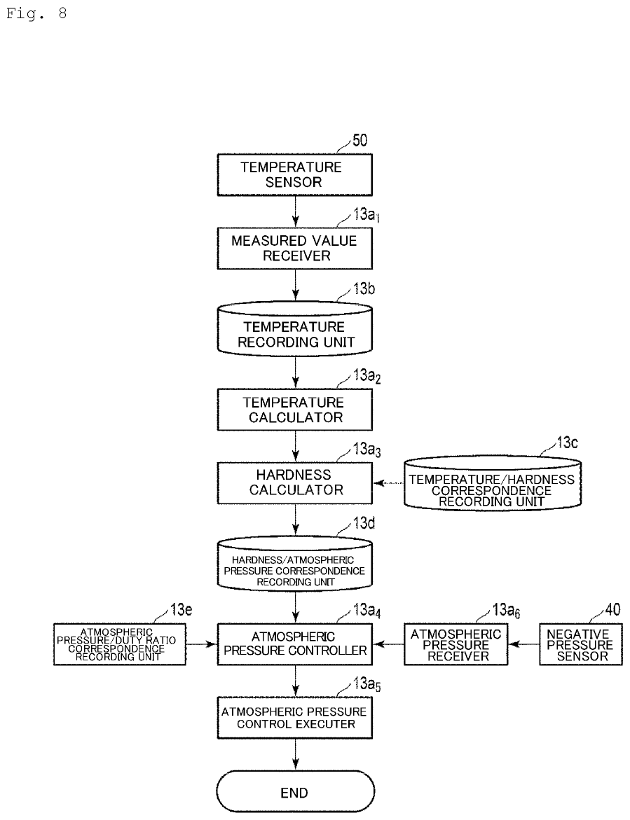

FIG. 10 is a block diagram illustrating pseudo-food texture presentation processing realized by the pseudo-food texture presentation device illustrated in FIG. 9.

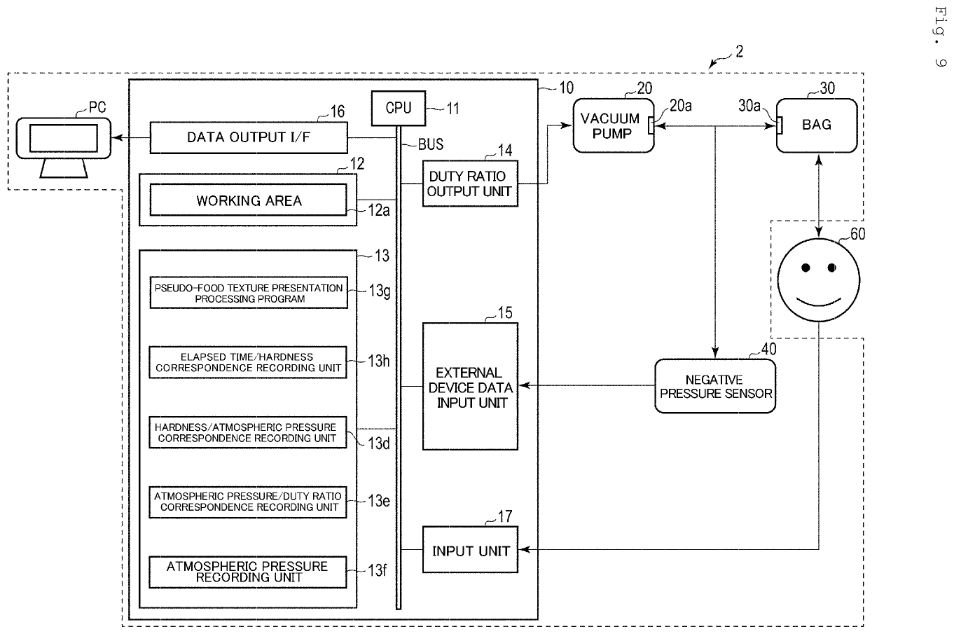

FIG. 11 is a block diagram illustrating a configuration of a pseudo-food texture presentation device according to Second embodiment.

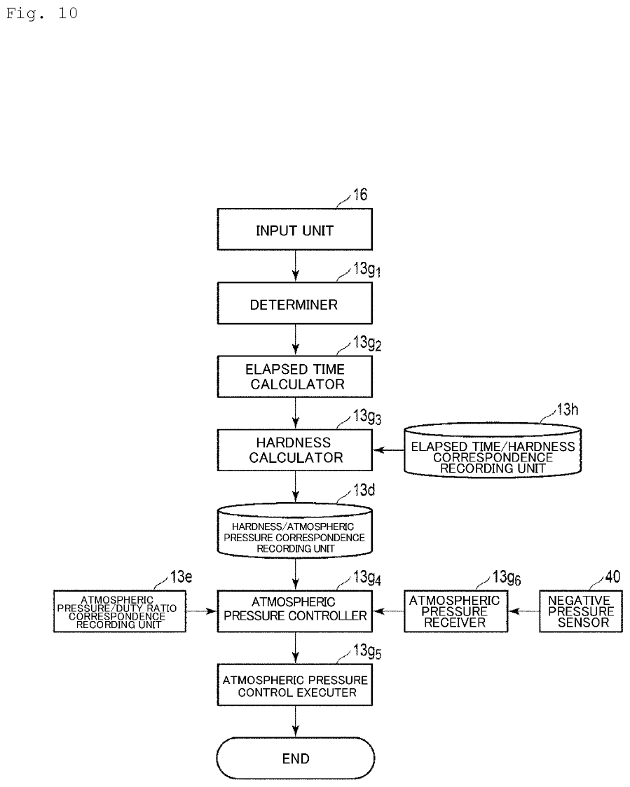

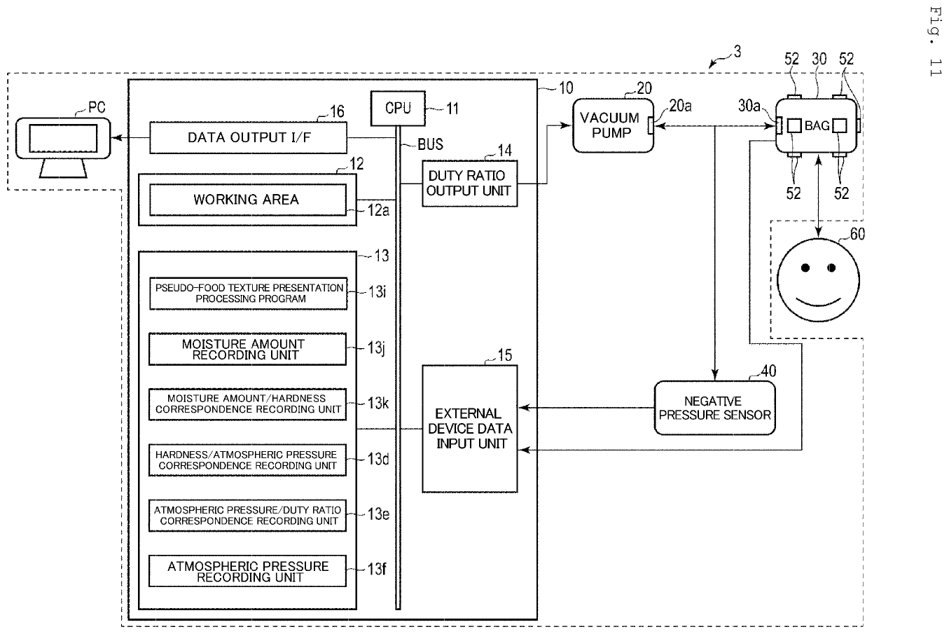

FIG. 12 is a block diagram illustrating pseudo-food texture presentation processing realized by the pseudo-food texture presentation device illustrated in FIG. 11.

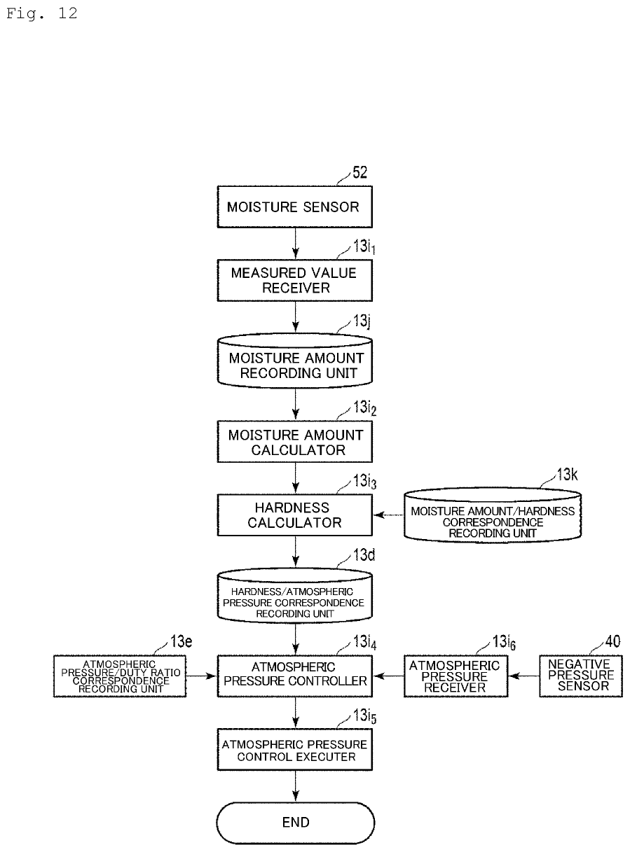

FIG. 13 is a diagram illustrating a result of hardness sensory evaluation at each stage by a Scheffe's paired comparison method.

### DESCRIPTION OF EMBODIMENTS

Hereinafter, an embodiment of the present invention will be described with reference to the drawings.

### First Embodiment

### (Configuration)

FIG. 1 is a perspective view schematically illustrating an outer appearance of a pseudo-food texture presentation device 1 according to First embodiment. As illustrated in FIG. 1, the pseudo-food texture presentation device 1 includes a microcomputer 10, a vacuum pump 20, a bag 30 which is an enclosure body, a negative pressure sensor 40, a plurality of temperature sensors 50, and a personal computer PC.

The vacuum pump 20 includes a suction opening 20a used to suck air. The suction opening 20a and an air opening 30a of the bag 30 are connected by, for example, a flexible tube GT1 made of polyurethane and a flexible tube GT2 formed of an acrylic pipe and a silicon hose via a junction JC. The vacuum pump 20 sucks air inside the bag 30 to generate a negative pressure inside the bag 30.

A motor is provided inside the vacuum pump 20. An air amount sucked from the bag 30 is adjusted (controlled) by controlling a duty ratio of a pulse width modulation (PWM) signal for driving the motor and controlling a revolution speed of the motor.

The bag 30 is, for example, formed of a deformable (elastic) substance such as rubber or silicon in a bag shape (in this example, a rubber balloon) and, for example, contains a granular material such as coffee grounds or a starch. The hardness of the bag 30 is chard variously in accordance with an air amount (the degree of vacuum) sucked. by the vacuum pump 20. When air is sucked by the vacuum pump 20 and the bag 30 is contracted, the density of the granular material increases. Thus, the hardness of the bag 30 increases. Lower atmospheric pressure inside the bag 30 results in higher hardness of the bag 30. In this way, by controlling the density of the granular material inside the bag 30, it is possible to control the hardness of the bag 30 by a jamming transition.

The negative pressure sensor 40 is connected to the junction JC via, for example, the flexible tube GT2 and has a function of sensing the atmospheric pressure inside the bag 30.

The plurality of temperature sensors 50 are disposed at a plurality of positions on the surface of the bag 30 (hereinafter also referred to as sensor positions). In this example, the temperature sensors 50 are disposed on the outer surface of the bag 30 and measure the outside temperature of the bag 30 at each sensor position, that is, a temperature of an environment in which the bag 30 is present (hereinafter also referred to as ambient environment of the bag 30). Here, the outer surface of the bag 30 is a surface on the outside of the bag 30. Specifically, the outer surface of the bag 30 is a surface touched by tissues such as the teeth, the tongue, and the like of a user 60 who puts the bag 30 in her or his mouth.

The temperature sensors 50 measure the outside temperature of the bag 30 and transmit, to the microcomputer 10, temperature data including measured values of the temperature and measurement times at which the temperature is measured. The temperature sensors 50 obtain the measured values at predetermined time intervals (for example, intervals of 10 milliseconds) and output the temperature data in real time.

It is desirable to provide the plurality of temperature sensors 50 on the bag 30 as in the embodiment, but it is also possible to provide only one temperature sensor 50 on the bag 30.

The microcomputer 10 is connected to the vacuum pump 20 the negative pressure sensor 40, the plurality of temperature sensors 50, and the personal computer PC by signal lines S. The present invention is not limited to wired communication and wireless communication may be used.

The personal computer PC includes a display unit D such as a liquid crystal display device and has a function of receiving various kinds of measured data (for example, measured data such as atmospheric data and temperature data inside the bag 30) from the microcomputer 10 and outputting the measured data or graphs or The like obtained by processing the measured data to the display unit D.

FIG. 2 is a block diagram schematically illustrating a configuration of a microcomputer 10 in the pseudo-food texture presentation device 1. As illustrated in FIG. 2, the microcomputer 10 includes a central processing unit (CPU) 11 serving as a control unit, a random access memory (RAM) 12, a recording unit 13, a duty ratio output unit 14, an external device data input unit 15, and a data output interface (I/F) 16. The CPU 11 is connected to the random access memory (RAM) 12, the recording unit 13, the duty ratio output unit 14, the external device data input unit 15, and the data output interface 16 via a system bus BUS.

The RAM 12 includes a working area 12a.

The recording unit 13 is configured from a hard disk, a flash memory, or the like and includes a program area in which a pseudo-food texture presentation processing program 13a is stored, a temperature recording unit 13b, a temperature/hardness correspondence recording unit 13c, a hardness/atmospheric c pressure correspondence recording unit 13dan atmospheric pressure/duty ratio correspondence recording unit 13e, and an atmospheric pressure recording unit 13f .

The pseudo-food texture presentation processing program 13a is executed by the CPU 11 using the working area 12aWhen the pseudo-food texture presentation processing program 13a is executed by the CPU 11, pseudo-food. texture presentation processing to be described below is performed by the CPU 11.

The temperature recording unit 13b records temperatures measured by the plurality of temperature sensors 50 in association with measurement times which are times at which the measurement is performed.

The temperature/hardness correspondence recording unit 13c pre-records a correspondence table (database) in which a temperature corresponds to hardness of the bag 30. The correspondence table is set such that, for example, the hardness of the bag 30 is lower as the temperature is higher.

The hardness of the bag 30 can be set in seven stages in accordance with an atmospheric pressure (0 [kPa] to −60 [kPa]) inside the bag 30 which can be realized by sucking of the vacuum pump 20. For example, stage 1 is hardness at an atmospheric pressure of 0 [kPa], stage 2 is hardness at an atmospheric pressure of −10 [kPa], stage 3 is hardness at an atmospheric pressure of −20 [kPa], stage 4 sis hardness at an atmospheric pressure of −30 [kPa], stage 5 is hardness at an atmospheric pressure of −40 [kPa], stage 6 is hardness at an atmospheric pressure of −50 [kPa], and stage 7 is hardness at an atmospheric pressure of −60 [kPa]. For example, a lower stage corresponds to lower hardness of the bag 30, and thus a soft food texture. A higher stage corresponds to higher hardness of the bag 30, and thus a hard food texture. In other words, the hardness of the bag 30 is the lowest at stage 1. The hardness of the bag 30 becomes higher as the stage increases through stages 2, 3, . . . , and 6. Thus, at stage 7, the hardness of the bag 30 is the highest.

FIG. 3 is a diagram illustrating an example of the correspondence table recorded. on the temperature/hardness correspondence recording unit 13c. As illustrated in FIG. 3, when T is a temperature, for example, the hardness is set at stage 7 for T<T₁, the hardness is set at stage 6 for T₁≤T₂, the hardness is set at stage 5 for T₂≤T<T₃, the hardness is set at stage 4 for T₃≤T<T₄, the hardness is set at stage 3 for T₄≤T<T₅, the hardness is set at stage 2 for T₅≤T<T₆, and the hardness is set at stage 1 for T₆≤T. Here, specific temperatures [°C.] are set at T₁ to T₇.

The seven stages described above may not all be used. For example, as will be described below with reference to FIG. 13, a combination of the stages at which a difference in hardness can be perceived at the time of mastication may used. For example, the hardness is set at stage 7 for T<T₁₁, the hardness is set at stage 4 for T₁₁≤T<T₁₂, the hardness is set at stage 2 for T₁2 ≤T<T₁₃, and the hardness is set at stage 1 for T₁₃≤T. Here, specific temperatures [° C.] are set at T₁₁, T₁₂, and T₁₃.

The present invention is not limited to the seven stages and the hardness of the bag 30 may be set in fewer or more stages, By setting the hardness of the bag 30 in many stages, it is possible to continuously change the hardness substantially. As a result, it is possible to smoothly change food texture presented to a user.

Referring to FIG. 2, the hardness/atmospheric pressure correspondence recording unit 13d pre-records a correspondence table (database) in which each stage of the hardness of the bag 30 corresponds to an atmospheric pressure at which the hardness at each stage is obtained. FIG. 4 is a diagram illustrating an example of the correspondence table recorded on the hardness/atmospheric pressure correspondence recording unit 13d, In the example illustrated in FIG. 4, stage 1 corresponds to 0 [kPa], stage 2 corresponds to −10 [kPa], stage 3 corresponds to −20 [kPa], stage 4 corresponds to −30 [kPa], stage 5 corresponds to −40 [kPa], stage 6 corresponds to −50 [kPa], and stage 7 corresponds to −60 [kPa].

Referring to FIG. 2, the atmospheric pressure/duty ratio correspondence recording unit 13e pre-records a correspondence table (database) in which an atmospheric pressure inside the bag 30 corresponds to a duty ratio of the PWM signal. FIG. 5 is a diagram illustrating an example of a correspondence table recorded on the atmospheric pressure/duty ratio correspondence recording unit 13e. As illustrated in FIG. 5, an atmospheric pressure inside the bag 30 monotonically decreases with respect to a duty ratio of a PWM signal.

Instead of the correspondence table illustrated in FIG. 5, the atmospheric pressure/duty ratio correspondence recording unit 13e may pre-record a correspondence table in which seven values of duty ratios [%] are described in association with seven pressure values (that is, 0 [kPa], −10 [kPa], . . . , −60 [kPa]) included in the hardness/atmospheric pressure correspondence recording unit 13d.

Referring to FIG. 2, the atmospheric pressure recording unit 13f records an atmospheric pressure inside The bag 30 measured by the negative: pressure sensor 40 in real time.

The duty ratio output unit 14 outputs a PWM signal with the duty ratio set by the CPU 11 to the vacuum pump 20. Thus, a suction amount (the degree of vacuum) sucked from the bag 30 by the vacuum pump 20 is adjusted (controlled).

The external device data input unit 15 receives atmospheric pressure data indicating a measured value of the atmospheric pressure inside the bag 30 from the negative pressure sensor 40 and also receives temperature data indicating a measured value of the temperature from the plurality of temperature sensors 50.

The data output interface 16 outputs, to the personal computer PC, various kinds of data such as data regarding the temperature measured by the temperature sensors 50 in addition to the data regarding the atmospheric pressure inside the bag 30 measured by the negative pressure sensor 40. Specifically, the CPU 11 outputs, to the personal computer PC via the data output interface 16, various kinds of data stored in the temperature recording unit 13b, the temperature/hardness correspondence recording unit 13c, the hardness/atmospheric pressure correspondence recording unit 13d, the atmospheric pressure/duty ratio correspondence recording unit 13e, and the atmospheric pressure recording unit 13f included in the recording unit 13. Thus, not only the various kinds of data but also tables, graphs, or the like obtained by processing the various kinds of data are displayed on the display unit D of the personal computer PC.

The pseudo-food texture presentation device 1 that has the foregoing configuration realizes pseudo-food texture presentation processing to be described below when the CPU 11 controls an operation of each constituent element in response to a command described in the pseudo-food texture presentation processing program 13a.

The shape of the bag 30 is not limited to the shape illustrated in FIG. 1. The bag 30 can be changed to another shape such as a rectangular parallelepiped. That is, the shape can also be presented as food texture to a user in a pseudo-manner along with the hardness.

FIGS. 6A to 6D are diagrams illustrating aspects in which an atmospheric pressure inside the bag 30 is controlled by the vacuum pump 20. Specifically, FIG. 6A is a diagram illustrating a state of the bag 30 when an atmospheric pressure inside the bag 30 is 0 [kPa]. FIG. 6B is a diagram illustrating a state of the bag 30 when an atmospheric pressure inside the bag 30 is −10 [kPa]. FIG. 6C is a diagram illustrating a state of the bag 30 when an atmospheric pressure inside the bag 30 is −30 [kPa]. FIG. 6D is a diagram illustrating a state of the bag 30 when an atmospheric pressure inside the bag 30 is −60 [kPa].

As illustrated in FIGS. 6A to 6D, when the atmospheric pressure inside the bag 30 is changed from 0 [kPa], the hardness of the bag 30 is changed and it remains in the same shape as in a state in which the atmospheric pressure inside the bag 30 is 0 [kPa].

In this way, by changing the atmospheric pressure (the stage of hardness) inside the bag 30, the user 60 can be allowed to perceive another hardness of the bag 30 that has substantially the same shape.

A method of changing the shape of the bag 30 and presenting the changed shape to the user will be described with reference to FIGS. 7A to 7D.

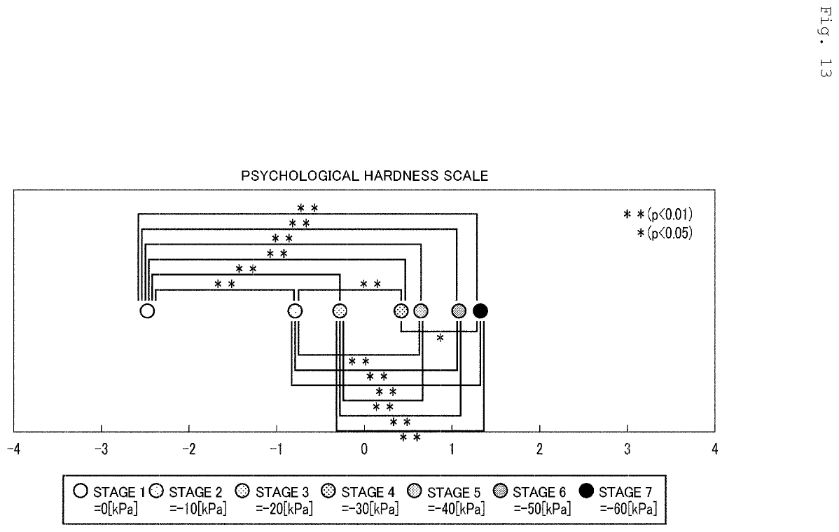

First, as illustrated in FIG. 7A, an atmospheric pressure inside the bag 30 is assumed to be 0 [kPa]. As illustrated in FIG. 7B, a person who is an operator deforms the shape of the bag 30 with her or his hand in this state. Thereafter, as illustrated in FIG. 7C, the shape of the bag 30 is kept by causing the vacuum pump 20 to generate a negative pressure (for example, −5 [kPa]) inside the bag 30. As illustrated in FIG. 70, when the user puts the bag 30 in her or his mouth, the atmospheric pressure inside the bag 30 is adjusted to an initial state (for example, −60 [kPa]).

In this way, food texture of food in a rectangular parallelepiped of which the hardness is changed with the temperature can be presented to the user 60.

(Operation)

FIG. 8 is a diagram schematically illustrating pseudo-food texture presentation. processing according to the Embodiment. The pseudo-food texture presentation processing illustrated in FIG. 8 is realized. when the CPU 11 in the microcomputer 10 executes the pseudo-food texture presentation processing program 13a using the working area 12a. The pseudo-food texture presentation processing program 13a causes the CPU 11 to function as a measured value receiver 13a₁, a temperature calculator 13a₂, a hardness calculator 13a₃, an atmospheric pressure controller 13a₄, and an atmospheric pressure control executer 13a₅.

The measured value receiver 13a₁ receives the temperature data from the plurality of temperature sensors 50 via the external device data input unit 15 and records the received temperature data on the temperature recording unit 13b.

The temperature calculator 13a₂ calculates a temperature of the ambient environment of the bag 30 based. on the temperature data received by the measured value receiver 13a₁. As an example, the temperature calculator 13a₂ may calculate an average value obtained by averaging measured values (latest measured values) of the temperatures measured at the plurality of sensor positions as the temperature of the ambient environment of the bag 30. As another example, the temperature calculator 13a₂ may calculate a highest measured value among the measured values (latest measured values) of the temperatures measured at the plurality of sensor positions as the temperature of the ambient environment of the bag 30.

The hardness calculator 13a₃ calculates the hardness of the bag 30 in accordance with the temperature calculated by the temperature calculator 13a₂ with reference to the correspondence table recorded on the temperature/hardness correspondence recording unit 13c. For example, when a temperature Tc calculated by the temperature calculator 13a₂ is in the range of T₃≤Tc<T₄, the hardness calculator 13a₃ determines stage 4 as the hardness of the bag 30 at the temperature Tc by referring to the correspondence table illustrated in FIG. 3.

Based on the temperature calculated by the temperature calculator 13a₂, the atmospheric pressure controller 13a₄ calculates an atmospheric pressure at which the hardness of the bag 30 calculated by the hardness calculator 13a₃ is obtained from the correspondence table recorded in the hardness/atmospheric pressure correspondence recording unit 13d. Subsequently, the atmospheric pressure controller 13a₄ sets a duty ratio of the PWM, signal in accordance with the calculated atmospheric pressure at the calculated atmospheric pressure by referring to the correspondence table recorded on the atmospheric pressure/duty ratio correspondence recording unit 13e. For example, when the hardness calculator 13a₃ determines stage 4 as the hardness of the bag 30, the atmospheric pressure controller 13a₄ determines −30 [kPa] as the atmospheric pressure at which the hardness at stage 4 is obtained by referring the correspondence table illustrated in FIG. 4 at stage 4. Then, the atmospheric pressure controller 13a₄ determines 6 [%] as the duty ratio at which the atmospheric pressure inside the bag 30 is set to −30 [kPa] by referring to the correspondence table illustrated in FIG. 5.

Finally, to adjust (control) the atmospheric pressure inside the bag 30, the atmospheric pressure control executer 13a₅ controls a revolution speed of the motor of the vacuum pump 20 based on the duty ratio set by the atmospheric pressure controller 13a₄. Specifically, the atmospheric pressure control executer 13a₅ generates a PWM signal with the duty ratio set by the atmospheric pressure controller 13a₄ supplies the generated PWM signal to the vacuum pump 20 via the duty ratio output unit 14.

In this way, for example, when. the temperature of the ambient environment of the bag 30 is determined to increase the CPU 11 may reduce the duty ratio of the PWM signal. That is, the CPU 11 may reduce the hardness of the bag 30 with the increase in the temperature of the ambient environment of the bag 30 in the order from stages 7 to 1. Thus, it is possible to present a user with food texture of food (for example, ice cream) which becomes soft with an increase in temperature.

The pseudo-food texture presentation processing program 13a may cause the CPU 11 to further function as an atmospheric pressure receiver 13a₆. In this case, the atmospheric pressure receiver 13a₆ may receive the measured value of the atmospheric pressure inside the bag 30 from the negative pressure sensor 40 and the atmospheric pressure controller 13a₄ may adjust the duty ratio based on the measured value received by the atmospheric pressure receiver 13a₆ so that the atmospheric pressure inside the bag 30 becomes the atmospheric pressure at which the hardness calculated by the hardness calculator 13a₃ is calculated.

(Effects)

In this way, the pseudo-food texture presentation device 1 monitors the temperature of the ambient temperature of the bag 30 using the temperature sensors 50 mounted on the bag 30. The pseudo-food texture presentation device 1 may reduce the density of the granular material inside the bag 30, for example, as the temperature of the ambient environment of the bag 30 increases. The hardness of the bag 30 is reduced by a jamming transition by reducing the density of the granular material inside the bag 30. In the embodiment, the density of the granular material inside the bag 30 is controlled by changing the atmospheric pressure inside the bag 30. Thus, it is possible to present the user with food texture such as overall continuous softening texture, for example, food texture of an ice cream, when the temperature increases.

### MODIFICATION EXAMPLES

In the above-described example, the temperature sensors 50 are disposed on the outer surface of the bag 30. However, the temperature sensors 50 may be disposed on the inner surface of the bag 30. Here, the inner surface of the bag 30 is a surface on the inner side of the bag 30. Specifically, the inner surface of the bag 30 is a surface regulating an inner space in which the granular material is sealed.

In an example in which the temperature sensors 50 are disposed on. the inner surface of the bag 30, the temperature calculator 13a₂ calculates a temperature of the bag 30 itself. Since a method of calculating the temperature of the bag 30 based on the measured values of the temperatures obtained by the temperature sensors 50 is similar to the method of calculating the temperature of the ambient environment of the bag 30, as described above, description thereof will be omitted.

The correspondence table recorded on the temperature/hardness correspondence recording unit 13c may be set so that the higher temperature results in higher hardness of the bag 30. In this way, it is possible to present the user with food texture of food hardened with an increase in temperature.

Instead of the temperature sensor 50, a humidity sensor maybe used. When the humidity sensor is used, the CPU 11 monitors a humidity of the ambient environment of the bag 30 and controls the density of the granular material inside the bag 30 with a change in humidity of the ambient environment of the bag 30.

In the above-described example, an enclosure body is configured as one bag 30. However, the enclosure body may be configured as a plurality of bags in which a granular material is sealed. The enclosure bodies maybe formed in a structure in which a plurality of bags are disposed in parallel, for example, so that bunches of citrus fruits or the like are lined up (a first structure) or may be in a multiple structure in which a plurality of bags are disposed in a nesting shape (a second structure). For example, in a multiple structure by two bags, one bag (an outer bag) covers the entire other bag (an inner bag). In the second structure, by controlling the hardness for each bag, it is possible to express food texture of food that has different food textures on outer and inner sides of fondant chocolate, for example.

The enclosure body may have a structure in which the first and second structures are combined (a third structure). For example, the enclosure body includes first, second and third bags, the first bag covers the entire second and third bags, and the second and third bags are disposed in parallel in the first bag. In the third structure, for example, it is possible to express food texture in which citrus fruits of lined-up bunches are inside fondant .chocolate

When the enclosure body is configured as a plurality of bags, a granular material may be changed for each bag. For example, different granular materials may be used for one bag among the plurality of bags and another bag among the plurality of bags. For example, when the enclosure body includes first and second bags, coffee grounds may be sealed in the first bag and starch may be sealed in the second bag. For example, when the enclosure body includes first, second, and third bags, coffee grounds may be sealed in the first and second bags and starch may be sealed in the third bag.

When the enclosure body is configured as a plurality of bags, a material of the bag may be changed for each bag. For example, when the enclosure body includes first and second bags, the first bag may be formed of natural rubber and the second bag may be formed of silicon. For example, when the enclosure body includes first, second, and third bags, the first and second bags may be formed of natural rubber and the third bag may be formed of silicon.

When the enclosure body is configured as a plurality of bags, the density of a granular material is controlled for each bag. Thus, it is possible to present a user with food texture with different hardness for each part, specifically, food texture in which only a part with a high temperature is soft.

The density of the granular body may be controlled in at least one of the plurality of bags. In other words, the density of the granular body may not be controlled in one bag or several bags among the plurality of bags.

Further, by partitioning the bag 30 into a plurality of pieces and controlling the density of the granular material for each piece, it is possible to present a user with food textures with different hardness for each piece.

The pseudo-food texture presentation device 1 may further have a density measurement function (a density sensor) of measuring the density of the granular material sealed inside the bag 30. For example, the density measurement function is capable of measuring the density of the granular material by pre-ascertaining the contents of the granular material inside the bag 30, a volume (capacity) of the bag 30 in an initial state, and an air amount remaining in the bag 30 and measuring the volume (capacity) of the air discharged from the bag 30 or flowing in the bag 30.

When the granular material flows in and out from the bag 30 with the discharge and inflow of air, the amount of the granular material inside the bag 30 is changed. By considering a change in an amount of air remaining in the bag 30 along with inflow or outflow of the granular material, it is possible to accurately measure the density of the granular material inside the bag 30.

The CPU 11 of the pseudo-food texture presentation device 1 can calculate the hardness of the bag 30 based on the measured density of the granular material.

In the above-described example, the density of the granular material inside the bag 30 is changed by controlling the atmospheric pressure inside the bag 30 using the vacuum pump 20. However, the density of the granular material may be changed by providing a structure in which the granular material flows in the bag 30 or flows out from the bag 30 and controlling the amount of the granular material which is inside the bag 30.

Further, by combining the vacuum pump and the inflow or outflow of the granular material, it is possible to also change an increase or decrease the volume (size) and the shape of the bag 30 while keeping the hardness (or the density of the granular material) of the bag 30 or change the hardness while keeping the shape of the bag 30.

### Second Embodiment

### (Configuration)

FIG. 9 is a block diagram schematically illustrating a configuration of a pseudo-food texture presentation device 2 according to Second embodiment. As illustrated in FIG. 9, the pseudo-food texture presentation device 2 includes the microcomputer 10, the vacuum pump 20, the bag 30, the negative pressure sensor 40, and the personal computer PC. In FIG. 9, similar reference signs are given to similar constituent elements to the constituent elements illustrated in FIG. 2 and description of these constituent elements will be omitted.

The microcomputer 10 includes the CPU 11, the RAM 12, the record unit 13, the duty ratio output unit 14, the external device data input unit 15, the data output interface (I/F) 16, and the input unit 17. The CPU 11 is connected to the random access memory (RAM) 12, the recording unit 13, the duty ratio output unit 14, the external device data input unit 15, the data output interface 16, and the input unit 17 via the system bus BUS.

The recording unit 13 includes a program area in which a pseudo-food texture presentation processing program 13g is stored, an elapsed time/hardness correspondence recording unit 13h, the hardness/atmospheric pressure correspondence recording unit 13d, the atmospheric pressure/duty ratio correspondence recording unit 13e, and the atmospheric pressure recording unit 13f.

The pseudo-food texture presentation processing program 13g is executed by the CPU ail using the working area 12a. When the pseudo-food texture presentation processing program 13g is executed by the CPU 11, pseudo-food texture presentation processing to be described below is performed by the CPU 11.

The elapsed time/hardness correspondence recording unit 13h pre-records a correspondence table (database) in which an elapsed time which is a time elapsed from a standard time corresponds to the hardness of the bag 30. In the embodiment, the standard time is a time at which the bag 30 is in the mouth of the user 60. The correspondence table may be set such that, for example, the hardness of the bag 30 is lower as the elapsed time increases. Since the correspondence table can be generated by a similar method to the method of generating the correspondence table recorded on the temperature/hardness correspondence recording unit 13c described in First embodiment, specific description thereof will be omitted.

The input unit 17 is, for example, a hardware button and is used for the user 60 to input, to the pseudo-food texture presentation device 2, the fact that the bag 30 is in the user's mouth. The user presses the button at a timing at which the bag 30 is in the user's mouth. The input unit 17 is not limited to the button and may be another input device such as a microphone. When the input unit 17 is a microphone, the user inputs the fact that the bag 30 is in the user's mouth by voice.

The input unit 17 may be provided at a different location from the casing of the microcomputer 10, for example, in the personal computer PC.

The pseudo-food texture presentation device 2 that has the foregoing configuration realizes pseudo-food texture presentation processing to be described subsequently by causing the CPU 11 to control an operation of each constituent element in response to a command described in the pseudo-food texture presentation processing program 13g.

(Operation)

FIG. 10 is a diagram schematically illustrating pseudo-food texture presentation processing according to the embodiment. The pseudo-food texture presentation processing illustrated in FIG. 10 is realized when the CPU 11 executes the pseudo-food texture presentation processing program 13g. The pseudo-food texture presentation processing program 13g causes the CPU 11 to function as a determiner 13g₁, an elapsed time calculator 13g₂, a hardness calculator 13g₃, an atmospheric pressure controller 13g₄, and an atmospheric pressure control executer 13g₅.

The determiner 13g₁ determines whether the bag 30 is in the mouth of the user 60 in response to an operation performed by the user 60 on the input unit 17. In an example in which the user 60 presses a button when the bag 30 is in the mouth of the user 60, it is determined that the bag 30 is in the user's mouth when the determiner 13g₁ detects that the button is pressed. When the determiner 13g₁ detects that the button is pressed again, the determiner 13g₁ may determine that the bag 30 is taken out from her or his mouth of the user 60.

When the determiner 13g₁ determines that the bag 30 is in the mouth of the user 60, the elapsed time calculator 13g₂ calculates an elapsed time. Specifically, the elapsed time calculator 13g₂ sets, as a standard time, a time at which the determiner 13g₁ determines that the bag 30 is in the mouth of the user 60 and calculates the elapsed time from the standard time.

The hardness calculator 13g₃ calculates the hardness of the bag 30 in accordance with the elapsed time calculated by the elapsed time calculator 13g₂ with reference to the correspondence table recorded on the elapsed time/hardness correspondence recording unit 13h.

The atmospheric pressure controller 13g₄ and the atmospheric pressure control executer 13g₅ perform same processing as that of the above-described atmospheric pressure controller 13a₄ and the atmospheric pressure control executer 13a₅ (see FIG. 8). Therefore, description of the atmospheric pressure controller 13g₄ and the atmospheric pressure control executer 13g₅ will be omitted.

The pseudo-food texture presentation processing program 13g may cause the CPU 11 to further function as an atmospheric pressure receiver 13g₆. The atmospheric pressure receiver 13g₆ performs the same processing as the above-described atmospheric pressure receiver 13a₆ (see FIG. 6). Therefore, description of the atmospheric pressure receiver 13g₆ will be omitted.

That is, the processing performed to control the density of the granular material inside the bag 30 to realize the hardness calculated by the hardness calculator 13g₃ is common between First and Second embodiments.

(Effects)

As described above, the pseudo-food texture presentation device 2 monitors, as an elapsed time, a time in which the bag 30 is in the mouth of the user 60. The pseudo-food texture presentation. device 2 may reduce the density of the granular material inside the bag 30, for example, as the elapsed time is longer. The hardness of the bag 30 is reduced by a jamming transition by reducing the density of the granular material inside the bag 30. Thus, it is possible to present the user with food texture such as overall continuous softening texture, for example, food texture of an ice cream, when the time is elapsed.

### MODIFICATION EXAMPLES

In the above-described example, based on an input by the user, it is determined that the bag 30 is in the user's mouth. A method of determining whether the bag 30 is in the user's mouth is not limited to the foregoing example.

For example, a humidity sensor is disposed on the bag 30 and the determiner 13g₁ may perform the determination based on an outside humidity of the bag 30 measured by the humidity sensor. Specifically, the determiner 13g₁ determines that the bag 30 is in the user's mouth when a measured value of humidity is less than a threshold at time t and is equal to or greater than the threshold at time t+. At difference between times t and t+1 is, for example, 10 milliseconds. The determiner 13g₁ may determine that the bag 30 is taken out from the user's mouth when the measured value of humidity is equal to or greater than the threshold at time t and is less than the threshold at time t+1. Instead of the humidity sensor, a temperature sensor or a moisture sensor may be used.

When it is determined that the bag 30 is in the user's mouth, the CPU 11 may control the density of the granular material inside the bag 30, in this example, the atmospheric pressure inside the bag 30. When the determination is performed using a humidity sensor or a temperature sensor, it is determined that the bag 30 is in the user's mouth after the bag 30 is in the user's mouth. Therefore, by controlling the density of the granular material inside the bag 30 when it is determined that the bag 30 is in the user's mouth, it is possible to change the hardness of the bag 30 immediate after the bag 30 is the mouth of the user 60.

The standard time is not limited to the time at which the bag 30 is in the mouth of the user 60. The standard time may be a time before the bag 30 is in the mouth of the user 60. That is, the elapsed time may be included in not only a period in which the bag 30 is in the mouth of the user 60 but also a period in which the bag 30 is not in the mouth of the user 60.

A temperature sensor or a humidity sensor may be provided on the bag 30 and the density of the granular material inside the bag 30 may be controlled based on a cumulative value in accordance with an elapsed time of a temperature or humidity. In this case, the CPU 11 identifies, as the standard time, a time at which the bag 30 is in the mouth of the user 60 by one of the above-described method and calculates a cumulative value (a cumulative value over elapsed time) of measured values of the temperature or humidity from the standard time to a current time. Then, the CPU 11 calculates the hardness of the bag 30 in accordance with the calculated cumulative value of the measured values of the temperature or humidity and controls the density of the granular material inside the bag 30, as described above. In this way, by controlling the density of the granular material based on two indexes of the elapsed time and the temperature or humidity, it is possible to improve reproduction of overall continuous softening food texture as in an ice cream.

For example, when the bag 30 is in the mouth of the user 60 and opens her or his mouth wide, the temperature or humidity of the ambient environment of the bag 30 temporarily decreases in some cases. In this case, the hardness of the bag 30 is temporarily returned (increased). By performing the control based on the cumulative value, it is possible to prevent the hardness of the bag 30 from being temporarily returned.

### Third Embodiment

### (Configuration)

FIG. 11 is a block diagram schematically illustrating a configuration of a pseudo-food texture presentation device 3 according to Third embodiment. As illustrated in FIG. 11, the pseudo-food texture presentation device 3 includes the microcomputer 10, the vacuum pump 20, the bag 30, the negative pressure sensor 40, a plurality of moisture sensors 52, and the personal computer PC. In FIG. 11, similar reference signs are given to similar constituent elements to the constituent elements illustrated in FIG. 2 and description of these constituent elements will be omitted.

The plurality of moisture sensors 52 are each disposed at a plurality positions (sensor positions) on the surface of the bag 30. The moisture sensors 52 are disposed on the outer surface of the bag 30. The moisture sensors 52 measure a moisture amount (a saliva amount) attached to the sensor positions and transmit measured values of the moisture amounts and moisture amount data including measurement times to the microcomputer 10. The moisture sensors 52 obtains measured values at predetermined time intervals (for example, 10 millisecond intervals) and output the moisture amount data in real time.

It is preferable to provide the plurality of moisture sensors 52 according to the embodiment on the bag 30, but only one moisture sensor 52 may be provided on the bag 30.

The recording unit 13 in the microcomputer 10 includes a program area in which a pseudo-food texture presentation processing program 13i is stored, a moisture amount recording unit 13j, a moisture amount/hardness correspondence recording unit 13k, a hardness/atmospheric pressure correspondence recording unit 13d, the atmospheric pressure/duty ratio correspondence recording unit 13e, and the atmospheric pressure recording unit 13f.

The pseudo-food texture presentation processing program 13i is executed by the CPU 11 using the working area 12a. When the pseudo-food texture presentation processing program 13i is executed by the CPU 11, pseudo-food. texture presentation processing to be described below is performed by the CPU 11.

The moisture amount recording unit 13j records the moisture amounts measured by the plurality of moisture sensors 52 in association with measurement times.

The moisture amount/hardness correspondence recording unit 13k pre-records a correspondence table (database) in which a moisture amount attached to the bag 30 corresponds to the hardness of the bag 30. The correspondence table may be set such that, for example, the hardiness of the bag 30 is lower as the moisture amount increases. Since the correspondence table can be generated by a similar method to the method of generating the correspondence table recorded on the temperature/hardness correspondence recording unit 13c described in First embodiment, specific description thereof will be omitted.

(Operation)

FIG. 12 is a diagram schematically illustrating pseudo-food texture presentation processing according to the embodiment. The pseudo-food texture presentation processing illustrated in FIG. 12 is realized when the CPU 11 executes the pseudo-food texture presentation. processing program 13i. The pseudo-food texture presentation processing program 13i causes the CPU 11 to function as a measured value receiver 13i₁, a moisture amount calculator 13i₂, a hardness calculator 13i₃, an atmospheric pressure controller 13i₄, and an atmospheric pressure control executer 13i₅.

The measured value receiver 13i₁ receives moisture amount data from the plurality of moisture sensors 52 via the external device data input unit 15 and records the received moisture amount data on the moisture amount recording unit 13j.

The moisture amount calculator 13i₂ calculates a moisture amount attached to the bag 30 based on the moisture amount data received by the measured value receiver 13i₁. As an example, the moisture amount calculator 13i₂ may calculate a sum value obtained by summing measured values (latest measured values) of the moisture amount measured at the plurality of sensor positions as the moisture amount attached to the bag 30. As another example, the moisture amount calculator 13i₂ may calculate a highest measured value among the measured values (latest measured values) of the moisture amount measured at the plurality of sensor positions as the moisture amount attached to the bag 30.

The hardness calculator 13i₃ calculates the hardness of the bag 30 in accordance with the moisture amount calculated by the moisture amount calculator 13i₂ with reference to the correspondence table recorded on the moisture amount/hardness correspondence recording unit 13k.

The atmospheric pressure controller 13i₄ and the atmosphere pressure control executer 13i₅ perform the same processing as the above-described atmospheric pressure controller 13a₄ and the atmospheric pressure control executer 13a₅ (see FIG. 6). Therefore, description of the atmospheric pressure controller 13i₄ and the atmospheric pressure control executer 13i₅ will be omitted.

The pseudo-food texture presentation processing program 13i may cause the CPU 11 to further function as an atmospheric pressure receiver 13i₆. The atmospheric pressure receiver 13i₆ perform the same processing as the above-described atmospheric pressure receiver 13a₆ (see FIG. 6). Therefore, description of the atmospheric pressure receiver 13i₆ will be omitted.

That is, the processing performed to control the density of the granular material inside the bag 30 to realize the hardness calculated by the hardness calculator 13i₃ is common between First and Third embodiments.

(Effects)

As described above, the pseudo-food texture presentation device 3 monitors a moisture (saliva) amount attached to the bag 30. The pseudo-food texture presentation device 3 may reduce the density of the granular material inside the bag 30, for example, as the moisture amount attached to the bag 30 is larger. The hardness of the bag 30 is reduced by a jamming transition by reducing the density of the granular material inside the bag 30. Thus, it is possible to present the user with food texture such as overall continuous softening texture as a saliva amount attached to food increases.

### MODIFICATION EXAMPLES

In the above-described example, the enclosure body is configured as one bag 30. Instead of this, the enclosure body may be configured as the plurality of bags 30. The enclosure body may have any one structure among the above-described first, second, and third structures.

For example, when the enclosure body has the first structure, the plurality of moisture sensors 52 are disposed on the plurality of bags 30, respectively. The CPU 11 calculates the attached moisture amount for each bag 30 and controls the hardness for each bag 30 based on a calculation result. Thus, it is possible to present the user with food texture such as softening texture in only a part to which much saliva is attached.

The moisture amount calculator 13i₂ may calculate a cumulative value obtained by accumulating the measured values of the moisture amounts from the standard time to the current time, that is, a cumulative value of the moisture amounts over the elapsed time, for each moisture sensor 52 and may calculate a sum of the cumulative values in the moisture sensors 52 per each sensor as a moisture amount attached to the bags 30. The moisture amount calculator 13i₂ may calculate a cumulative value obtained by accumulating the measured values of the moisture amounts from the standard time to the current time for each moisture sensor 52 and may calculate the largest value among the cumulative values in the moisture sensors 52 as the moisture amount attached to the bag 30.

As a method of calculating the standard time or the elapsed time, a similar method to the method described in Second embodiment can be used. For example, it may be determined that the bag 30 is in the mouth of the user 60 based on comparison between a threshold and a moisture amount attached to the bag 30 and measured by a moisture sensor provide on the bag 30, and that time may be set as the standard time. In the embodiment, based on a relative position or the like of the bag 30 to the mouth of the user 60, it may be determined that the bag 30 is in the rnouth of the user 60.

In the above-described embodiments, the index indicating an environment in which there is the bag 30 is measured, such as the temperature of the ambient environment of the bag 30, the humidity of the ambient environment of the bag 30, the temperature of the bag 30, the moisture amount attached to the bag 30, and the elapsed time from the time at which the bag 30 is in the mouth of the user 60. Then, the density of the granular material inside the bag 30 is controlled based on the measured index and the hardness of the bag 30 is controlled using the jamming transition. Thus, it is possible to present a user with food texture in a pseudo-manner.

(Others)

In First embodiment, the seven stages are used as the hardness of the bag 30. A method of identifying a combination of stages in which a person can perceive a difference in the hardness between the seven stages when a person masticates the bag 30 will be described. A similar method can also be applied to an operation (for example, an action of putting the bag 30 between a tongue and the upper jaw) of the mouth of a user on the bag 30 other than mastication.

First, an experiment (1. Experiment conditions, 2. Experiment method, and 3. Experiment result) in which a subject is allowed to masticate the bag 30 will be described. By referring to a result obtained from this experiment, it is possible to identify whether a person can perceive the difference in the hardness between any two stages in her or his mouth.

1. Experiment Conditions

The atmospheric pressure inside the bag 30 is changed in the range of 0 [kPa] to −60 [kPa] and the hardness of the bag 30 is changed from stages 1 to 7 by causing the vacuum pump 20 to suck air inside the bag 30.

In an experiment method to be described below, the hardness between two stages (for example, between stages 1 and 2) set by the vacuum pump 20 is compared. In the comparison of the hardness, a Scheffe's paired comparison method is used. That is, a subject is allowed to masticate the bags 30 with two different types of hardness (for example, the hardness of stage 1 and the hardness of stage 2) in sequence to assess a level and the level is assessed by determining how much the stack is hard. All the stages in which the bags are assessment targets are combined.

Two different types of hardness may be generated using one bag 30 and the subject may be allowed to masticate the bag 30 with the two types of hardness in sequence. Two bags 30 with different types of hardness may be prepared and the subject may be allowed to masticate the two bags 30 with the different types of hardness in sequence.

Specifically, for example, the hardness at stage 1 and the hardness at stage 2 are compared to assess how much the bags are hard at which stages, for example, levels 1 to 5. When the levels are assessed in combination of stages 1 to 7, the levels are assessed in combination of ₇C₂=21 in total.

That is, the levels are assessed in combination of stages 1 and 2, stages 1 and 3, . . . , and stages 1 to 7. Subsequently, the levels are assessed in combination of stages 2 and 3, stages 2 and 4, . . . , and stages 2 and 7. Further, the levels are assessed in combination of stages 3 and 4, stages 4 and 5, . . . , and stages 5 and 6. Finally, the levels are assessed in combination of stages 6 and 7.

Here, the five levels are assumed to be (i) “the first masticated bag is very harder”, (ii) “the first masticated bag is slightly harder”, (iii) “the first and second masticated bags are substantially the same hard”, (iv) “the second masticated bag is slightly harder”, and (v) “the second masticated bag is very harder”. The levels (i) to (v) are converted into scores (numerical values)

Specifically, with regard to the score at the first stage, the score i s converted into “4” when (i) is selected, the score is converted into “2” when (ii) is selected. Similarly, the score is converted into “0” when (iii) is selected, the score is converted into “−2” when (iv) is selected, and the score is converted into “−4” when (v) is selected. Conversely, with regard to the score at the second stage, the score is converted into “−4” when (i) is selected, and the score is converted into“−2” when (ii) is selected. Similarly, the score is converted into “0” when (iii) is selected, the score is converted into “2” when (iv) is selected, and the score is converted into “4” when (v) is selected. That i s, when a comparison target is perceived. to be hard, the score at each stage becomes larger.

In this way, the level assessment of relative hardness at each stage to a comparison stage is converted into the score. The score conversion is performed on the assessment of the foregoing 21 methods.

In This way, a numerical value obtained by averaging the scores at each stage is plotted as the relative score at each stage. A plotting result will be described below with reference to FIG. 13.

2. Experiment Method

In the experiment, the subject practices a job of the following (2-1) to (2-4) once. When the practice ends, steps of (2-1) to (2-4) are performed by the 21 methods.

(2-1) The user masticates the bag 30 of which the hardness is kept at the first stage by the vacuum pump 20 so that the following conditions (A) to (C) are satisfied for 10 seconds (this is referred to as first mastication).

(A) The mastication is performed between the upper front and back teeth on the right when viewed from the user 60.

(B) The mastication is performed near the middle of the bag 30.

(C) The mastication is performed as usual when the user always eats.

(2-2) When the mastication ends, the user waits for 10 seconds. While the user waits, the user equally extends the contents of the bag.

(2-3) The user masticates the bag 30 of which the hard is kept at the second stage by the vacuum pump 20 so that the foregoing conditions (A) to (C) are satisfied for 10 seconds (this is referred to as second mastication).

(2-4) At the time of ending, k subjects (where k is a natural number) assess the levels (i) to (v) to determine which bag is harder by comparing the first mastication (2-1) with the second mastication (2-3).

3. Experiment Result

FIG. 5 illustrates an experiment. result. FIG. 5 is a diagram (a graph) illustrating a result of sensory assessment at each step plotted by the Scheffe's paired comparison method. The sensory assessment is assessing characteristics of a target (herein, the hardness of the bag 30) using the senses of the user.

First, a method of reading measured data in the graph will be described. A relative score at each stage is plotted on the horizontal axis (a psychological hardness scale) of the graph. The scores are results obtained when the k subjects assess the level of the relative hardness in combination of the 21 methods. Therefore, the score at each stage is plotted as a relative score on the psychological hardness scale. The horizontal axis of the graph represents the psychological hardness scale, 4 is a relative score indicating that the subject feels the hardest, and −4 is a relative score indicating that the subject feels the softest.

That is, for example, when stage 1 is seen from stage 2, the k subjects feel very soft at stage 1. As a result, when seen from stage 2, a difference from stage 1 is about “−1.7” on the psychological scale.

On the other hand, when the hardness of the bag 30 is increased at stage 5, stage 6, and stage 7, it can be known that the bags with the hardness at stages 5, 6, and 7 have different hardness from stage 2 when seen from stage 2. However, between stages 5, 6, and 7, the difference in the hardness is not felt so much. As a result, a difference between stages 5 and 6 on the psychological scale is about “0.4” and a difference between stages 6 and 7 is about “0.2” which are small values.

Then, for example, the subject feels considerably soft when the subject masticates the bag 30 with the hardness of stage 1 when seen from stage 7 than when the subject masticates the bag 30 of stage 7. As a result, a difference between stages 1 and 7 on the psychological scale is about “−3.8”.

Here, “*” or “**” is attached on a solid line between the stages, above the solid line, or below the solid line. The solid line, “*,” and “**” indicate that “there is a certain constant significant difference” between two stages (for example, stages 1to 2). The “significant difference” is not a difference caused by chance or due to an error and is a “meaningful difference”.

For example, stages 1 and 2 are connected by a solid line and “** (p<0.01)” is assigned. Further, for example, stages 4 and 7 are connected by a solid line and “* (p<0.05)” is assigned.

Here, p is a p value. The p value is an index used to determine whether the significant difference arises. When the p value is a value lower than a significant level (normally, 5%), it is determined that there is a significant difference. Further, the significant level is a probability serving as a standard at which a probability of a certain event arising is determined to be rarely an accident (to be significant). That is, when the p value obtained at the time of occurrence of a certain event (for example, a result that (i) “the first masticated bag is very hard” in the stage assessment at stages 1 and 2) is equal to or less than 5%, a measurement result obtained from the event is rarely considered to be an accident, that is, it is determined that there is a significant difference.

In this experiment, in the level assessment in which stage 1 and stages 2 to 7 are combined, stage 2 and stages 4 to 7 are combined, and stage 3 and stages 5 to 7 are combined, the p value=0.01 or less (< significant level=1%) is obtained. Therefore, it is proved that the user 60 can perceive the difference in the hardness in her or his mouth at a probability of 99% or more.

In the assessment in which stages 4 and 7 are combined, 0.01<p value<0.05 is obtained. Therefore, it is proved that the user 60 can perceive the difference in the hardness in her or his mouth at a probability of 9% or more.

In this way, in this experiment, it is proved that the user 60 can perceive the difference in the hardness between stage 1 and stages 2 to 7, between the stage 2 and stages 4 to 7, between stage 3 and stages 5 to 7, and between stages 4 and 7.

A case in which the hardness of the bag 30 is changed from stage 7 to a lower stage will be considered. With reference to FIG. 13, there are the following five patterns when the hardness of the bag 30 is changed between. stages at which the subject can perceive the difference in the hardness.

(1) Stages 7, 4, 2, and 1

(2) Stages 7, 4, and 1

(3) Stages 7, 3, and 1

(4) Stages 7, 2, and 1

(5) Stages 7 and 1

When the hardness of the bag 30 is changed in accordance with pattern (1), a change in food texture (hardness) can be presented to the user at the four stages. In patterns (2) to (4), the change i. the food texture can be presented to the user at the three stages. In pattern (5), the change in the food texture can be presented to the user at the two stages.

Further, for example, when the hardness of the bag 30 is changed from stage 6 to a lower stage, the change in the food texture can be presented at a maximum of three stages.

Accordingly, it is preferable to use pattern (1) above in which the change in the food texture can be expressed at the most stages.

Similarly, when the hardness of the bag 30 is changed to a higher stage, it is preferable to use the pattern in which a stage such as stage 1, 2, 4, or 7 is changed.

In the above-described embodiments, the density of the granular material inside the bag 30 (the atmospheric pressure inside the bag 30) may be controlled so that the hardness of the bag 30 is changed between stages at which the difference in the hardness can be perceived. The density of the granular material inside the bag 30 may be controlled so that the hardness of the bag 30 is changed simply step by step (for example, stages 7, 6, 5, . . . , and 1).

The hardness of the bag 30 at each stage depends on a material, a thickness, or a shape of the bag 30, the granular material, the amount of the granular material, or the like. Therefore, the foregoing combinations are exemplary combinations in which the stages at which the person can perceive the difference in the hardness when the person masticates the bag 30.

Next, a method of generating the database recorded on the atmospheric pressure/duty ratio correspondence recording unit 13e will be described.

1. Procedure

(1) The duty ratio s changed from 0 [%] to 100 [%] (normally turned on). p (2) After each duty ratio is designated and 2 seconds has passed, an atmospheric pressure [kPa] inside the bag 30 is acquired 100 times an total at intervals of 10 [ms].

(3) An average value of an atmospheric pressure [kPa] inside the bag 30 acquired 100 times is calculated.

2. Result

By performing the foregoing procedure, as illustrated in FIG. 5, the database indicating a relation between the duty ratio of the PWM signal for driving the vacuum pump 20 and the atmospheric pressure inside the bag 30 is obtained.

The present invention is not limited to the foregoing embodiments and various modifications can be made in execution steps within the scope of the present invention without departing from the gist of the present invention. Further, various inventions can be extracted by appropriately combining the plurality of constituent elements disclosed including the invention of the various steps in the foregoing embodiments. For example, when several constituent elements are deleted from all the constituent elements described in the embodiments or several constituent elements are combined but the problems mentioned in Technical Problem can be solved and the effects described in Effects of the Invention can be obtained, a configuration in which the constituent elements are deleted or combined can be extracted as an invention.

### REFERENCE SIGNS LIST

1 Pseudo-food texture presentation device
10 Microcomputer
11 CPU
12 RAM
12a Working Area
13 Recording unit
13a Pseudo-food texture presentation processing program
13a₁ Measured value receiver
13a₂ Temperature calculator
13a₃ Hardness calculator
13a₄ Atmospheric pressure controller
13a₅ Atmospheric pressure control executer
13a₆ Atmospheric pressure receiver
13b Temperature recording unit
13c Temperature/hardness correspondence recording unit
13d Hardness/atmospheric pressure correspondence recording unit
13e Atmospheric pressure/duty ratio correspondence recording unit
13f Atmospheric pressure recording unit
14 Duty ratio output unit
15 External device data input unit
16 Data output interface
20 Vacuum pump
20a Suction opening
30 Bag
30a Air opening
40 Negative pressure sensor
50 Temperature sensor
60 User
GT1, GT2 Flexible tube
2 Pseudo-food texture presentation device
13g pseudo-food texture presentation processing program.
13g₁ Determiner
13g₂ Elapsed time calculator
13g₃ Hardness calculator
13g₄ Atmospheric pressure controller
13g₅ Atmospheric pressure control executer
13g₆ Atmospheric pressure receiver
13h Elapsed time/hardness correspondence recording unit
17 Input unit
3 Pseudo-food texture presentation device
13i Pseudo-food texture presentation processing program
13i₁ Measured value receiver
13i₂ Moisture amount calculator
13i₃ Hardness calculator
13i₄ Atmospheric pressure controller
13i₅ Atmospheric pressure control executer
13i₆ Atmospheric pressure receiver
13j Moisture amount recording snit
13k Moisture amount/hardness correspondence recording unit
52 Moisture sensor

1. A pseudo-food texture presentation device comprising: an enclosure body in which a granular material is enclosed; a measurer configured to measure at least one index among a temperature of an ambient environment of the enclosure body, a humidity of the ambient environment of the enclosure body, a temperature of the enclosure body, and a moisture amount on a surface of the enclosure body; and an enclosure body controller configured to control density of the granular material inside the enclosure body in accordance with the measured index.

2. The pseudo-food texture presentation device according to claim 1, wherein the enclosure body controller controls the density of the granular material inside the enclosure body when the measured index exceeds a preset threshold.

3. The pseudo-food texture presentation device according to claim 1, wherein the enclosure body controller controls the density of the granular material inside the enclosure body in accordance with a cumulative value obtained by accumulating the measured index.

4. The pseudo-food texture presentation device according to any one of claims 1, further comprising: a determiner configured to determine whether the enclosure body is in a user's mouth, wherein the enclosure body controller controls the density of the granular material inside the enclosure body in accordance with the measured index and when the determiner determines that the enclosure body is in a user's mouth.

5. A pseudo-food texture presentation method performed by a computer, the method comprising: measuring at least one index among a temperature of an ambient environment of an enclosure body in which a granular material is enclosed, a humidity of the ambient environment of the enclosure body, a temperature of the enclosure body, and a moisture amount on a surface of the enclosure body; and
controlling density of the granular material inside the enclosure body in accordance with the measured index.

6 The pseudo-food texture presentation method according to claim 5, wherein the controlling of the density of the granular material inside the enclosure body includes controlling the density of the granular material inside the enclosure body when the measured index exceeds a preset threshold.

7. The pseudo-food texture presentation method according to claim 5, wherein the controlling of the density of the granular material inside the enclosure body includes controlling the density of the granular material inside the enclosure body in accordance with a cumulative value obtained by accumulating the measured index.

8. The pseudo-food texture presentation method according to any one of claims 5, further comprising: determining whether the enclosure body is in a user's mouth, wherein the controlling of the density of the granular material inside the enclosure body includes controlling the density of the granular material inside the enclosure body in accordance with the measured index and when it is determined that the enclosure body is in a user's mouth.

9. A non-transitory computer-readable medium having computer-executable instructions that, upon execution of the instructions by a processor of a computer, cause the computer to function as texture presentation device according to claim 1.
